(19) United States
(12) Patent Application Publication
(10) Pub. No.: US 2025/0011268 A1
(43) Pub. Date: Jan. 9, 2025

(54) PROCESS FOR PRODUCING DIMETHYL ETHER (DME) FROM SYNTHESIS GAS

(71) Applicant: L'Air Liquide, Societe Anonyme pour l'Etude et l’Exploitation des Procedes Georges Claude, Paris (FR)

(72) Inventors: Stephane HAAG, Frankfurt am Main (DE); Frank CASTILLO-WELTER, Frankfurt am Main (DE); Thomas RENNER, Frankfurt am Main (DE); Lin LIN, Frankfurt am Main (DE)

(73) Assignee: L'Air Liquide, Societe Anonyme pour l'Etude et l’Exploitation des Procedes Georges Claude, Paris (FR)

(21) Appl. No.: 18/764,241

(22) Filed: Jul. 4, 2024

(30) Foreign Application Priority Data
Jul. 6, 2023 (EP) 23183874

(51) Int. Cl. C07C 41/05; B01D 53/66; B01J 8/04; C07C 41/34

(52) U.S. Cl. CPC C07C 41/05; B01D 53/66; B01J 8/0492; B01J 8/0496; C07C 41/34; B01D 2257/108; B01D 2257/50; B01J 2208/00628

(57) ABSTRACT

The invention relates to a process for producing dimethyl ether (DME). The invention provides that a first and a second reaction zone in which catalyst fills are arranged between two adjacent pillow plates and are traversable by the respective input gas are arranged in a common synthesis reactor. The pillow plates are traversable by a fluid cooling medium. The DME-containing product gas stream exiting the synthesis reactor is resolved into a DME end product stream, a gas byproduct stream containing unconverted carbon oxides and hydrogen, a methanol byproduct stream and a wastewater stream. The gas byproduct stream is at least partially returned to the reactor inflow to increase the altogether achieved DME yield.

### CROSS-REFERENCE TO RELATED APPLICATIONS

This application claims the benefit of priority under 35 U.S.C. § 119 (a) and (b) to EP patent application No. EP 23183874, filed Jul. 6, 2023, the entire contents of which are incorporated herein by reference.

### BACKGROUND

### Field of the Invention

The present invention relates to a process for producing dimethyl ether (DME) from a synthesis gas containing hydrogen and carbon oxides.

### Related Art

The catalytic production of dimethyl ether (DME) from methanol by catalytic dehydration has been known for many years. U.S. Pat. No. 2,014,408 thus describes a process for producing DME from methanol over catalysts such as aluminium oxide, titanium oxide and barium oxide, wherein temperatures of 350° C. to 400° C. are preferred.

Further information on the prior art and current practice in the production of dimethyl ether may be found in Ullmann's Encyclopedia of Industrial Chemistry, Sixth Edition, 1998 Electronic Release, keyword “Dimethyl Ether”. Especially Chapter 3 “Production” indicates that catalytic reaction of pure gaseous methanol is performed in a fixed bed reactor.

From a reaction engineering standpoint, catalytic dehydration of methanol to afford DME in the gas phase preferably employs fixed bed reactors since they are notable for their simplicity of design. Thus, German laid-open specification DE 3817816 describes a process, integrated in a methanol synthesis plant, for producing dimethyl ether by catalytic dehydration of methanol without preceding separation of the synthesis gas not converted in the methanol reactor. The dehydration reactor used is a simple fixed bed reactor. If said reactor is configured without additional measures for temperature control but rather is merely surrounded by an outer insulation for avoiding heat losses it is also known as an adiabatic fixed bed reactor.

The dehydration of methanol to afford dimethyl ether according to the reaction equation 2 CH₃OH═(CH₃)₂O+H₂O is an exothermic equilibrium reaction and it follows therefrom that from a thermodynamic standpoint high conversions are achieved at the lowest possible reaction temperatures. By contrast, from a standpoint of reaction kinetics a minimum reaction temperature is required to achieve sufficient reaction rates and thus acceptable methanol conversions. The disadvantage of the hitherto employed adiabatic fixed bed reactors is the inability to ensure optimal temperature management to ensure high conversions and to minimize the formation of byproducts.

The formation of byproducts, for example carbon monoxide CO, carbon dioxide CO₂, hydrogen H₂ and methane CH₄, occurs preferentially at higher temperatures. A suspected possible cause for the formation of the three first-mentioned byproducts is steam cracking of methanol in the input stream or previously formed DME with steam which is formed as a reaction byproduct. Methane may be formed, for example, by a subsequent reaction of the carbon oxides formed with hydrogen. The formation of these by-products is unwanted since they have an adverse effect on the purity of the reaction product and reduce the selectivity of the reaction for DME.

The theoretical study “Modeling and Optimization of MeOH to DME Isothermal Fixed-bed Reactor”, Farsi et al., International Journal of Chemical Reactor Engineering, Volume 8, 2010, Article A79, describes the optimal temperature profile of reactor temperature for the catalytic dehydration of methanol to afford dimethyl ether in a quasi-(or largely) isothermal fixed bed reactor in which the solid catalyst is arranged in tubes which have partially evaporating water flowing around them as cooling medium on the shell side. A genetic algorithm that takes into account thermodynamic and kinetic aspects of the dehydration reaction calculates a temperature profile decreasing exponentially from the reactor inlet to the reactor outlet as optimal, wherein the reactor inlet temperature is about 800 K and the reactor outlet temperature about 560 K. Based on this axial temperature profile a methanol conversion of about 86% is calculated for the optimized isothermal reactor while that of the adiabatic reactor is only about 82%. However, the aforementioned paper provides no information about how to configure the design of an optimized fixed bed reactor for DME production from methanol. In addition, only methanol conversion and not the formation of any byproducts is used as an optimization criterion.

A disadvantage of the previously discussed processes for producing DME is that they require methanol to be initially produced and isolated in costly and inconvenient fashion. Conversion of methanol to the target product DME is then carried out in a second step. Two-stage processes for DME synthesis altogether have the disadvantages that the required synthesis plant comprises two different reactors, thus necessitating a greater number of equipment positions at considerable capital expenditure. The methanol produced in the first synthesis step further requires removal, purification and cooling.

Processes for so-called DME direct synthesis, in which the intermediate product methanol does not occur and/or is not isolated but rather the synthesis proceeds from synthesis gas right through to the target product DME, have therefore already been discussed in the prior art. For example, US patent specification U.S. Pat. No. 10,501,394 B2 discloses a process for producing DME from synthesis gas where at least one stream of synthesis gas is subjected to at least one synthesis step and where the components present in the input stream are at least partially converted to dimethyl ether (DME) to obtain at least one raw product stream containing at least DME and the unconverted components of the input stream. The at least one synthesis step is thus performed under isothermal conditions.

However, such processes for single-step synthesis or DME direct synthesis have the following disadvantages:
The single-step synthesis produces many byproducts, for example methanol, methane, carbon dioxide, small amounts of C2+ hydrocarbons and unconverted synthesis gas constituents.
More unconverted compounds must be recycled to the synthesis reactor to ensure a high DME yield.
The selectivity of single-step synthesis for DME is typically poor.
The H₂/CO ratio of the synthesis gas has a great effect on selectivity.
The service life of the catalyst is often too short.

### SUMMARY OF THE INVENTION

It is accordingly an object of the present invention to propose a process for producing DME which avoids the recited disadvantages of single-step and two-step production processes hitherto known from the prior art. This object is achieved in a first aspect of the invention by a process having the features of claim 1. Further aspects of the invention are apparent from the dependent process claims.

The methanol synthesis conditions suitable for conversion of synthesis gas to methanol and the DME synthesis conditions suitable for conversion of methanol to DME are known to those skilled in the art from the prior art, for example the published specifications discussed at the outset. These are the physicochemical conditions under which a measurable, preferably industrially relevant, conversion of synthesis gas to methanol/of methanol to DME is achieved. Necessary adjustments of these conditions to the respective operational requirements will be made by those skilled in the art on the basis of routine experiments. Any specific reaction conditions disclosed may serve as a guide but should not be regarded as limiting in relation to the scope of the invention.

The solid, liquid and gaseous/vaporous states of matter mentioned should always be considered in relation to the local physical conditions that exist in the respective process step or in the respective plant component, unless stated otherwise. In the context of the present patent application, the gaseous and vaporous states of matter should be considered to be synonymous.

Thermal separation processes in the context of the present invention include all separation processes based on the establishment of a thermodynamic phase equilibrium. These preferably include distillation or rectification with in each case repeated adjustment of the vapour-liquid equilibrium or resolution of a gas-liquid mixture or vapour-liquid mixture in a phase separation apparatus with simple adjustment of the vapour-liquid equilibrium. However, the use of other thermal separation processes, for example absorption, extraction or extractive distillation, is also conceivable in principle.

In the context of the present invention a division or resolution/separation of a material stream is to be understood as meaning production of at least two substreams from the original material stream, wherein resolution/separation is associated with an intentional alteration of the composition of matter of the obtained substreams with respect to the original material stream, for example through application of a thermal separation process or at least thermal separation step to the original material stream. By contrast, division of the original material stream is generally not associated with a change in the composition of matter of the obtained substreams.

A pillow plate in the context of the invention is composed of two metal sheets (thermo sheets) which are welded together at the edges and over whose surface a multiplicity of spot welds, which likewise join the two plates to one another, are distributed. Such pillow plates may be produced by robots in automated fashion and thus very cost-effectively. After welding, the metal sheets are expanded by hydraulic forming, i.e. generally injection of a fluid under high pressure, thus creating pillow-like channels between the metal sheets which are freely traversable by a cooling or heating fluid. The pillow plates may be oriented in the synthesis reactor in parallel next to one another on the horizontal axis or orthogonally to the horizontal axis. The space between two pillow plates may be filled with a fill of a solid, granular catalyst. Appropriate arrangement of three or more pillow plates results in a sandwich like structure with good accessibility of the catalyst to inflowing and outflowing material streams, for example an input gas and a product gas, and very good heat transfer between the catalyst fills and the one cooling fluid or heating fluid.

The present invention provides for performing the two-step DME synthesis without isolation of the intermediate methanol in a common synthesis reactor in order to ideally obtain the advantages of both synthesis routes while minimizing the disadvantages. To this end a first reaction zone in which synthesis gas is converted into methanol is combined with a second reaction zone in which methanol is reacted further to afford DME. Both reaction zones are arranged in a common, pressure-bearing reactor shell (shell tube) and no isolation and purification of the intermediately produced methanol is carried out.

Both reaction steps are carried out over fills of a solid particulate catalyst specific to the respective partial reaction. The present invention provides that the catalyst fills are in each case arranged between two adjacent pillow plates and are traversable by the respective input gas. The pillow plates are traversable by a fluid cooling medium. The sandwich-like arrangement of catalyst layers and cooling layers results in very good thermal management within the catalyst bed with the result that isothermal conditions are achieved or at least approximated in the individual catalyst beds. So-called hotspots, i.e. zones of locally high temperature, are especially avoided or reduced in the catalyst beds. Hotspots have an adverse effect on the possible service life of the catalysts and in the case of exothermic equilibrium reactions reduce the conversion of the reactant components integrally achievable over a catalyst bed.

The DME-containing product gas stream exiting the synthesis reactor is passed to a separation apparatus operating according to at least one thermal separation process; this may be a single-step or multi-step distillation for example. The separation apparatus effects a resolution of the DME-containing product gas stream into a DME end product stream, a gas byproduct stream containing unconverted carbon oxides and hydrogen, a methanol byproduct stream and a wastewater stream. The gas byproduct stream is at least partially returned to the reactor inflow to increase the altogether achieved DME yield.

Since control of the reaction temperatures and of the pressure is important to achieve a maximum conversion, different embodiments of the invention are concerned with the utilization of different material streams as coolant and with the conduction thereof through the synthesis reactor. Coolants used may include special heat transfer fluids but also reactant and/or product streams of the reaction zones.

The pressure in the synthesis reactor according to the invention is about 40 to 90 bar absolute, preferably 60 to 80 bar absolute. Preference is given to a synthesis gas whose CO concentration is higher than its CO₂ concentration since this results in reduced water formation and improved selectivity for DME. However, other synthesis gases can also be used, for example including mixtures of CO₂ and hydrogen with little or no admixture of CO.

The temperature in the first reaction zone is preferably between 180° C. and 350° C., most preferably between 200° C. and 280° C.

It is preferable when the temperature in the second reaction zone is between 220° C. and 350° C., more preferably between 240° C. and 320° C., most preferably between 260° C. and 300° C. Studies have shown that high yields of DME and low yields of byproducts are achieved in these temperature ranges.

Coolants used may include special heat transfer fluids but also reactant and/or product streams of the reaction zones. In a first example water is used as cooling medium, wherein fresh water or water produced in the reaction or mixtures of both may be used for example. In a second example cold synthesis gas is used as cooling medium and is therefore itself preheated, thus reducing the heating energy demand of the process. In a third example a methanol byproduct stream from the separation apparatus is used as cooling medium. This reduces the energy demand of the downstream production of pure methanol.

An advantage of the process according to the invention is its fast startup capability. To this end, initially the second reaction zone (DME synthesis) is put into operation with methanol supplied from an external source and synthesis gas is passed through the second reaction zone as cooling medium in order to achieve the desired temperature and then allow the first reaction zone (methanol synthesis) to be put into operation.

It is advantageous in the process according to the invention when the synthesis reactor makes it possible to achieve minimization of byproducts while simultaneously achieving a near-equilibrium conversion (40% DME, 40% H₂O, 20% unconverted methanol). The compounds obtained as intermediates (methanol) and end products (DME, CO, CO₂, hydrogen and methane) are not catalyst poisons for the employed catalysts which are commercially available per se.

A maximum reaction temperature of 400° C. should not to be exceeded in the second reaction zone in order to minimize the extent of methanization.

### Further Aspects of the Invention

A second aspect of the process according to the invention is characterized in that a first portion of the gas byproduct stream is recycled to the DME synthesis reactor and introduced into the DME synthesis reactor together with the first input gas stream. This increases the conversion of reactant components and the DME yield.

A third aspect of the process according to the invention is characterized in that a second portion of the gas byproduct stream is discharged from the process as a purge stream. This prevents accumulation of any inert components in the context of the two partial reactions, for example of methane.

A fourth aspect of the process according to the invention is characterized in that at least a portion of the methanol byproduct stream is recycled to the DME synthesis reactor and introduced into the interspace and/or into the catalyst fills in the second reaction zone. This specifically makes it possible to increase the yield in the second partial reaction, the DME synthesis.

A fifth aspect of the process according to the invention is characterized in that a first cooling water stream is used as a first fluid cooling medium and a second cooling water stream is used as a second fluid cooling medium. This makes it easy to establish the optimal temperatures in the first and second reaction zone.

A sixth aspect of the process according to the invention is characterized in that a common cooling water stream is used as the first fluid cooling medium and as the second fluid cooling medium. This aspect allows resource-efficient use of cooling water.

A seventh aspect of the process according to the invention is characterized in that the common cooling water stream is initially passed through one reaction zone and then, after optional cooling, through the other reaction zone. This aspect permits resource-efficient use of cooling water and at the same time provides more options for establishing optimal temperatures.

An eighth aspect of the process according to the invention is characterized in that the common cooling water stream is initially passed through the second reaction zone and then, after optional cooling, through the first reaction zone. This aspect permits resource-efficient use of cooling water and at the same time provides more options for establishing optimal temperatures. The common cooling water stream then flows through the synthesis reactor in counter-current to the reactant and product gases.

A ninth aspect of the process according to the invention is characterized in that the common cooling water stream initially passes through the first reaction zone and then, after optional cooling, through the second reaction zone. This aspect permits resource-efficient use of cooling water and at the same time provides more options for establishing optimal temperatures. The common cooling water stream then flows through the synthesis reactor in co-current to the reactant and product gases.

A tenth aspect of the process according to the invention is characterized in that the first cooling water stream and/or the second cooling water stream and/or the common cooling water stream are run through the first reaction zone and/or the second reaction zone in co-current relative to the gas flow through the first reaction zone and/or the second reaction zone. This aspect relates to the flow direction of the cooling medium within a reaction zone.

An eleventh aspect of the process according to the invention is characterized in that the first cooling water stream and/or the second cooling water stream and/or the common cooling water stream are run through the first reaction zone and/or the second reaction zone in counter-current relative to the gas flow through the first reaction zone and/or the second reaction zone. This aspect relates to the flow direction of the cooling medium within a reaction zone.

A twelfth aspect of the process according to the invention is characterized in that after optional cooling at least a portion of the wastewater stream is used as the first cooling water stream and/or second cooling water stream and/or common cooling water stream. This aspect allow particularly resource-efficient use of cooling water.

A thirteenth aspect of the process according to the invention is characterized in that at least a portion of the first cooling water stream and/or the second cooling water stream and/or the common cooling water stream is at least partially evaporated upon passing through the first reaction zone and/or the second reaction zone and is discharged as a vapour or vapour-liquid biphasic mixture. Due to the phase change and the associated particularly large enthalpy change, a particularly large cooling effect is achieved.

A fourteenth aspect of the process according to the invention is characterized in that at least a portion of the first input gas stream and/or at least a portion of the gas byproduct stream recycled to the DME synthesis reactor is used as the first fluid cooling medium and/or as the second fluid cooling medium before the at least a portion of the first input gas stream and/or the at least a portion of the gas byproduct stream recycled to the DME synthesis reactor is introduced into the DME synthesis reactor. This saves cooling medium, for example cooling water or heat transfer oil, and the corresponding gas streams are pretempered.

A fifteenth aspect of the process according to the invention is characterized in that at least a portion of the methanol byproduct stream is introduced into the first reaction zone as the first fluid cooling medium, wherein the methanol byproduct stream is heated and then introduced into the interspace and/or into the catalyst fills in the second reaction zone. This specifically makes it possible to increase the yield in the second partial reaction, the DME synthesis. Cooling medium, for example cooling water or heat transfer oil, is simultaneously saved and the methanol byproduct stream is pretempered.

A sixteenth aspect of the process according to the invention is characterized in that initially the second reaction zone (DME synthesis) is put into operation with methanol supplied from an external source and in that synthesis gas is passed through the second reaction zone as cooling medium in order to achieve the desired temperature and then put the first reaction zone (methanol synthesis) into operation. This allows the process according to the invention to be put into operation particularly quickly.

A seventeenth aspect of the process according to the invention is characterized in that a maximum reaction temperature of 400° C. is not exceeded in the second reaction zone. This makes it possible to minimize the extent of the methanization.

An eighteenth aspect of the process according to the invention is characterized in that the temperature in the first reaction zone is between 180° C. and 350° C., most preferably between 200° C. and 280° C., and in that the temperature in the second reaction zone is between 220° C. and 350° C., more preferably between 240° C. and 320° C., most preferably between 260° C. and 300° C. Studies have shown that high yields of DME and low yields of byproducts are obtained in these temperature ranges.

A nineteenth aspect of the process according to the invention is characterized in that the operating pressure of the DME synthesis reactor is not less than 90 bar absolute and the minimum operating temperature of both reaction zones is not less than 250° C., preferably not less than 260° C. This allows the DME synthesis reactor to be operated without condensation occurring in the reactor interior, in particular inside the first and second reaction zone.

### BRIEF DESCRIPTION OF THE FIGURES

Further features, advantages and possible applications of the invention are apparent from the following description of working and numerical examples and from the drawings. All the features described and/or depicted, on their own or in any combination, form the subject-matter of the invention, irrespective of their combination in the claims or their dependency references.

In the figures:

FIG. 1 shows a schematic representation of the process according to the invention in an exemplary embodiment of a DME synthesis reactor,

FIG. 2 shows a detail view of a first reaction zone of the DME synthesis reactor according to the invention,

FIG. 3 shows a design detail of the pillow plates (schematic).

What is meant by “not shown” hereinafter is that an element in the figure under discussion is not graphically represented but nevertheless present in accordance with the description.

### DETAILED DESCRIPTION OF THE INVENTION

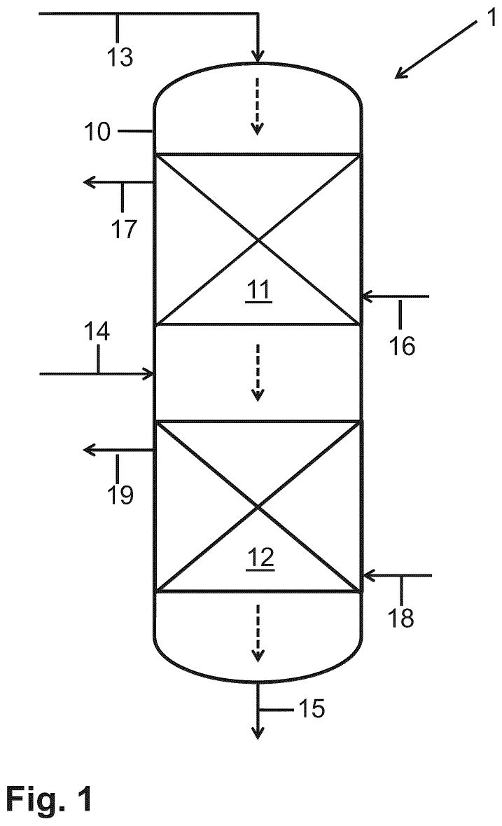

FIG. 1 shows a schematic representation of the process according to the invention in an exemplary embodiment of a DME synthesis reactor 1. In a common pressure-bearing shell tube 10 arranged vertically with respect to its longitudinal axis a first reaction zone 11 and a second reaction zone 12 are arranged one atop the other. Synthesis gas is introduced into the DME synthesis reactor 1 as an input stream via a conduit 13 and flows through said reactor from top to bottom; this is indicated by dashed flow arrows. The input stream enters the first reaction zone 11, whose catalyst zones are filled with a commercially available particulate catalyst active for methanol synthesis, via a first inlet (not shown).

In the first reaction zone 11 the input stream is at least partially converted into methanol under methanol synthesis conditions. The at least partially converted, methanol-comprising input stream is discharged from the first reaction zone 11 as a first, methanol-containing product gas stream via a first outlet (not shown) and then introduced into a second reaction zone 12 via a second inlet (not shown) for the first product gas.

In the second reaction zone 12 the first, methanol-containing product gas stream is at least partially converted into DME under DME synthesis conditions. This affords a second, DME-containing product gas stream which is discharged from the second reaction zone via a second outlet (not shown) and from the DME synthesis reactor 1 via a conduit 15 (product outlet) on the shell tube 10 and sent to a separation apparatus operating according to at least one thermal separation process (not shown).

The separation apparatus effects a resolution of the DME-containing product gas stream into a DME end product stream, a gas byproduct stream containing unconverted carbon oxides and hydrogen, a methanol byproduct stream and a wastewater stream. In one example the DME end product stream is supplied to a DME storage, DME final purification and/or DME further processing (all not shown). The gas byproduct stream is preferably at least partially recycled to the DME synthesis reactor 1 and introduced into said reactor via the conduit 13. The methanol byproduct stream is preferably at least partially recycled to the DME synthesis reactor 1, introduced into said reactor via a conduit 14 and sent directly to the second reaction zone. In one example the wastewater stream is discharged from the process. In a further example the wastewater stream is recycled to the DME synthesis reactor 1 as cooling medium after optional cooling.

A first fluid cooling medium is introduced into the first reaction zone 11 via a conduit 16 and an inlet (not shown). The first fluid cooling medium absorbs at least a portion of the reaction heat of the exothermic methanol synthesis and is thus itself heated. The heated first fluid cooling medium is discharged from the first reaction zone 11 via an outlet (not shown) and a conduit 17.

A second fluid cooling medium is introduced into the second reaction zone 12 via a conduit 18 and an inlet (not shown). The second fluid cooling medium absorbs at least a portion of the reaction heat of the exothermic DME synthesis from methanol and is thus itself heated. The heated second fluid cooling medium is discharged from the second reaction zone 12 via an outlet (not shown) and a conduit 19.

In one example the pressure in the synthesis reactor according to the invention is to 90 bar absolute, preferably 60 to 80 bar absolute. In one example preference is given to introducing into the DME synthesis reactor 1 a synthesis gas whose CO concentration is higher than its CO₂ concentration since this results in reduced water formation and improved selectivity for DME. However, other synthesis gases can also be used, for example including mixtures of CO₂ and hydrogen with little or no admixture of CO.

In one example the temperature in the first reaction zone 11 is preferably between 180° C. and 350° C., most preferably between 200° C. and 280° C. In one example the temperature in the second reaction zone 12 is between 220° C. and 350° C., more preferably between 240° C. and 320° C., most preferably between 260° C. and 300° C. Studies have shown that high yields of DME and low yields of byproducts are achieved in these temperature ranges.

Coolants used may include special heat transfer fluids but also reactant and/or product streams of the reaction zones. In one example water is used as cooling medium, wherein fresh water or water produced in the reaction or mixtures of both may be used for example. In a further example cold synthesis gas is used as cooling medium and is therefore itself preheated, thus reducing the heating energy demand of the process. In a further example a methanol byproduct stream from the separation apparatus is used as cooling medium. This reduces the energy demand of the downstream production of pure methanol.

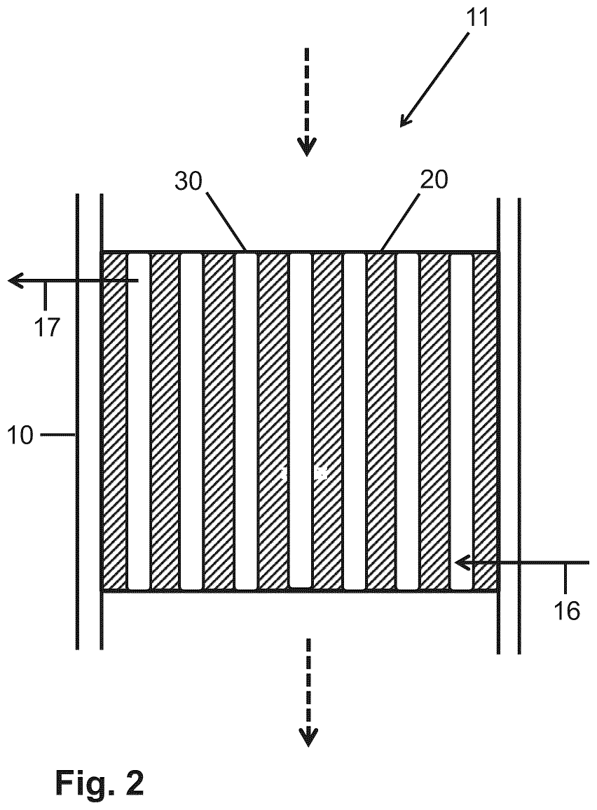

FIG. 2 shows a detail view of a first reaction zone 11 of the DME synthesis reactor 1 according to the invention. The second reaction zone 12 has fundamentally the same construction but the catalyst zones are filled with a catalyst active for the DME synthesis from methanol.

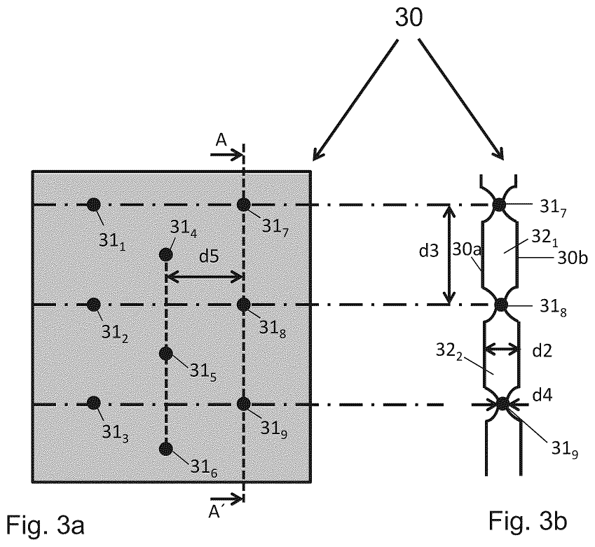

The first reaction zone 11 preferably has pillow plates 30 arranged in parallel and equally spaced apart. The detailed construction of the pillow plates 30 is elucidated below in connection with FIG. 3. The arrangement of the pillow plates results in interspaces 20 which are filled with fills of a solid, particulate catalyst active for the methanol synthesis. Both ends of the interspaces are permeable to gas streams. The lower end of the interspaces and preferably also the upper end of the interspaces comprise supports/retaining means (not shown) for the catalyst fills, for example sieve trays, perforated plates or wire mesh. The retaining means preferably attached at the inflow side of the gas flow into the first reaction zone 11 may therefore advantageously also be used to uniformize and distribute the input gas stream over the individual catalyst beds.

A first fluid cooling medium is introduced into the pillow plates 30 in the first reaction zone 11 via a conduit 16 and an inlet (not shown). The first fluid cooling medium absorbs at least a portion of the reaction heat of the exothermic methanol synthesis and is thus itself heated. The heated first fluid cooling medium is discharged from the first reaction zone 11 via an outlet (not shown) and conduit 17. Introduction and discharging of the first fluid cooling medium into the/from the pillow plates 30 is effected via a distributor system (not shown). This is indicated by the conduits 16 and 17 shown as arrows.

FIG. 3a shows an x-y view of a pillow plate 30 over the area of a metal sheet forming one side of the pillow plate 30. Dots 31₁ to 31₉ represent the so-called weld points with which the metal sheet is joined to the metal sheet (not shown) on the opposite side by an additional spot weld. Points 31₁ to 31₃, 31₃ to 31₆ and 31₆ to 31₉ each lie along a respective straight line and the points of every second straight line alternately lie along a straight line in the respective other dimension again. The straight lines run parallel to one another and have the spacing d5.

Since the pillow plate is welded not only at the edges of two superposed metal sheets but also has additional weld points 31₁ to 31₉ arranged on it the x-z view through a pillow plate gives the sectional view along the straight line A-A′ shown in FIG. 3b. Formed between the individual spot welds 31₁ to 31₉ are channels 32₁ and 32₂ which are generally produced by pressure forming, particularly preferably by hydroforming. The diameter d2 of such a channel 32₁ or 32₂ describes the spacing between the two metal sheets 30a and 30b at the maximum channel extent while the diameter d4 describes the thickness of the weld point 31₁ to 31₉. The spacing between two weld points 31₁ to 31₉, which also precisely corresponds to the spacing of two weld points 31₁ to 31₉ along a straight line, corresponds to d3. It is preferable when d3>d2>d4.

### NUMERICAL EXAMPLES

The following table summarizes exemplary operating conditions for the DME synthesis reactor according to the invention which allow reactor operation without falling below dew point temperature and thus without condensation in the reactor. This is important to ensure that uptime and potential service life of the catalyst is not impaired.

TABLE
Exemplary operating conditions for the DME synthesis reactor

| | Proportion/ | | Dew point/ | | Dew point/ |
|---|---|---|---|---|---|
| | mol % | p/bar | ° C. | p/bar | ° C. |
| Total | | 90.0 | | 60.0 | |
| pressure | | | | | |
| DME | 40.0 | 36.0 | | 24.0 | |
| Water | 40.0 | 36.0 | 250 | 24.0 | 225 |
| Methanol | 20.0 | 18.0 | | 12.0 | |

It is accordingly advantageous to select an operating pressure of the DME synthesis reactor according to the invention of not less than 90 bar absolute and a minimum operating temperature of both reaction zones of not less than 250° C., preferably not less than 260° C.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”: “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

### LIST OF REFERENCE NUMERALS

1 DME synthesis reactor
10 Shell tube
11 First reaction zone
12 Second reaction zone
13 Conduit (input gas stream)
14 Conduit (methanol side feed)
15 Conduit (product gas stream)
16 Conduit (first fluid cooling medium inlet)
17 Conduit (first fluid cooling medium outlet)
18 Conduit (second fluid cooling medium inlet)
19 Conduit (second fluid cooling medium outlet)
20 Catalyst fills in the interspaces between pillow plates (hatched)
30 Pillow plates
31 Spot welds
32 Free interior

1. A process for producing dimethyl ether (DME) from a synthesis gas containing carbon oxides and hydrogen comprising the steps of:
(a) providing a DME synthesis reactor comprising the following constituents:
(a1) a first reaction zone having a first inlet for synthesis gas as the first input gas and a first outlet for a first, methanol-containing product gas, wherein the first reaction zone comprises a multiplicity of pillow plates arranged such that:
a fill of a solid, particulate catalyst active for the methanol synthesis from the synthesis gas and traversable by the synthesis gas is in each case arranged between two adjacent pillow plates,
the pillow plates comprise an inlet for a first fluid cooling medium and an outlet for the first fluid cooling medium and in their interior are traversable by the first fluid cooling medium;
(a2) a second reaction zone having a second inlet for the first product gas and a second outlet for a second, DME-containing product gas, wherein the second reaction zone comprises a multiplicity of pillow plates arranged such that:
a fill of a solid, particulate catalyst active for the DME synthesis from methanol and traversable by the first product gas is in each case arranged between two adjacent pillow plates,
the pillow plates comprise an inlet for a second fluid cooling medium and an outlet for the second fluid cooling medium and in their interior are traversable by the second fluid cooling medium;
(a3) an outer, pressure-bearing shell tube having arranged in its interior the first reaction zone and, spaced apart therefrom by an interspace, the second reaction zone, wherein the shell tube comprises at the end adjacent to the first reaction zone a reactant inlet for introducing synthesis gas as the first input gas and at the end adjacent to the second reaction zone a product outlet for discharging the second, DME-containing product gas;
(b) introducing a synthesis gas stream as the first input gas stream into the DME synthesis reactor via the reactant inlet on the shell tube and into the first reaction zone via the first inlet;
(c) reacting the first input gas stream in the first reaction zone under methanol synthesis conditions;
(d) discharging a first methanol-containing product gas stream from the first reaction zone;
(e) introducing the first, methanol-containing product gas stream into the second reaction zone;
(f) reacting the first product gas stream in the second reaction zone under DME synthesis conditions;
(g) discharging a second, DME-containing product gas stream from the second reaction zone via the second outlet and from the DME synthesis reactor via the product outlet on the shell tube;
(h) supplying the DME-containing product gas stream to a separation apparatus operating according to at least one thermal separation process, resolving the DME-containing product gas stream in the separation apparatus into a DME end product stream, a gas byproduct stream containing unconverted carbon oxides and hydrogen, a methanol byproduct stream and a wastewater stream.

2. The process of claim 1, wherein a first portion of the gas byproduct stream is recycled to the DME synthesis reactor and introduced into the DME synthesis reactor together with the first input gas stream.

3. The process of claim 1, wherein a second portion of the gas byproduct stream is discharged from the process as a purge stream.

4. The process of claim 1, wherein at least a portion of the methanol byproduct stream is recycled to the DME synthesis reactor and introduced into the interspace and/or into the catalyst fills in the second reaction zone.

5. The process of claim 1, wherein a first cooling water stream is used as a first fluid cooling medium and a second cooling water stream is used as a second fluid cooling medium.

6. The process of claim 1, wherein a common cooling water stream is used as the first fluid cooling medium and as the second fluid cooling medium.

7. The process of claim 6, wherein the common cooling water stream is initially passed through one reaction zone and then, after optional cooling, through the other reaction zone.

8. The process of claim 7, wherein the common cooling water stream is initially passed through the second reaction zone and then, after optional cooling, through the first reaction zone.

9. The process of claim 7, wherein the common cooling water stream initially passes through the first reaction zone and then, after optional cooling, through the second reaction zone.

10. The process of claim 5, wherein the first cooling water stream and/or the second cooling water stream and/or the common cooling water stream are run through the first reaction zone and/or the second reaction zone in co-current relative to the gas flow through the first reaction zone and/or the second reaction zone.

11. The process of claim 5, wherein the first cooling water stream and/or the second cooling water stream and/or the common cooling water stream are run through the first reaction zone and/or the second reaction zone in counter-current relative to the gas flow through the first reaction zone and/or the second reaction zone.

12. The process of claim 1, wherein after optional cooling at least a portion of the wastewater stream is used as the first cooling water stream and/or second cooling water stream and/or common cooling water stream.

13. The process of claim 1, wherein at least a portion of the first cooling water stream and/or of the second cooling water stream and/or of the common cooling water stream is at least partially evaporated upon passing through the first reaction zone and/or the second reaction zone and is discharged as a vapour or vapour-liquid biphasic mixture.

14. The process of claim 1, wherein at least a portion of the first input gas stream and/or at least a portion of the gas byproduct stream recycled to the DME synthesis reactor is used as the first fluid cooling medium and/or as the second fluid cooling medium before the at least a portion of the first input gas stream and/or the at least a portion of the gas byproduct stream recycled to the DME synthesis reactor is introduced into the DME synthesis reactor.

15. The process of claim 1, wherein at least a portion of the methanol byproduct stream is introduced into the first reaction zone as the first fluid cooling medium, wherein the methanol byproduct stream is heated and then introduced into the interspace and/or into the catalyst fills in the second reaction zone.

16. The process of claim 1, wherein initially the second reaction zone (DME synthesis) is put into operation with methanol supplied from an external source and in that synthesis gas is passed through the second reaction zone as cooling medium in order to achieve the desired temperature and then put the first reaction zone (methanol synthesis) into operation.

17. The process of claim 1, wherein a maximum reaction temperature of 400° C. is not exceeded in the second reaction zone.

18. The process of claim 1, wherein the temperature in the first reaction zone is between 180° C. and 350° C., most preferably between 200° C. and 280° C., and in that the temperature in the second reaction zone is between 220° C. and 350° C., more preferably between 240° C. and 320° C., most preferably between 260° C. and 300° C.

19. The process of claim 1, wherein the operating pressure of the DME synthesis reactor is not less than 90 bar absolute and the minimum operating temperature of both reaction zones is not less than 250° C., preferably not less than 260° C.
